(19) United States
(12) Patent Application Publication
(10) Pub. No.: US 2013/0053837 A1
(43) Pub. Date: Feb. 28, 2013

(54) System and Method for Docking a Cornea with a Patient Interface Using Suction

(71) Applicants: Jochen Kandulla, Muenchen (DE); Roland Toennies, Gernlinden (DE); Markus Hailmann, Sachsenhausen (DE); Michael Tewes, Bruhl (DE)

(72) Inventors: Jochen Kandulla, Muenchen (DE); Roland Toennies, Gernlinden (DE); Markus Hailmann, Sachsenhausen (DE); Michael Tewes, Bruhl (DE)

(21) Appl. No.: 13/222,532

(22) Filed: Aug. 31, 2011

(51) Int. Cl. A61F 9/009; B23P 11/00

(52) U.S. Cl. 606/4; 137/15.01

(57) ABSTRACT

A system and method are provided for docking a patient interface device to the eye of a patient using suction. The patient interface device is formed by the structural cooperation of a base member and an attachment member, both of which have an open distal end. A contact lens is formed onto the open distal end of the base member, and a continuous abutment is formed onto the open distal end of the attachment member. When the interface device is placed onto the eye of a patient, only the continuous abutment contacts the sclera of the patient. An air pocket is formed between the contact lens and the continuous abutment. The air pocket is in fluid communication with a vacuum channel. A vacuum pump is then employed to suction the air from the air pocket and draw the surface of the eye into contact with the contact lens.

### FIELD OF THE INVENTION

The present invention pertains generally to systems and methods for performing ophthalmic surgery. More particularly, the present invention pertains to systems and methods for stabilizing an eye during ophthalmic surgery. The present invention is particularly, but not exclusively, useful as a system and method that draws the anterior surface of an eye into contact with a stabilizing lens to spread the cornea and thereby avoid creating wrinkles on the posterior surface of the cornea that would otherwise distort the laser beam being used for the ophthalmic surgery.

### BACKGROUND OF THE INVENTION

Surgical lasers are now commonly used in a variety of ophthalmic surgical procedures, including the treatment of ocular diseases and the correction of optical deficiencies. As an example, corneal reshaping procedures using lasers, such as the well known LASIK procedure, are now widely available. In these procedures, the surgical laser is often chosen as the tool of choice because of the ability of the laser to be accurately focused on extremely small amounts of ocular tissue. In addition, the ability of the laser to be guided to prescribed locations within the eye with precision and reliability has enabled a whole new class of ophthalmic procedures that require nothing short of pinpoint accuracy as the laser beam travels through various levels of the eye. Anatomical characteristics of the eye, however, can undermine the effectiveness of the laser procedure. In particular, this is so for ophthalmic laser surgery that is to be performed on tissue behind (i.e. posterior) the cornea. Specifically, it can happen that the beam of a laser can be significantly degraded by wrinkles that may be induced predominantly on the posterior surface of the cornea of an eye, when the eye is being stabilized by a contact lens. The effect of these wrinkles becomes most acute when the laser beam is used for procedures on tissues in the deeper regions of the eye beyond the cornea, such as the lens.

Typically, when an eye stabilizing device is required, it is placed against the anterior surface of the eye and pressed in a downward direction. As a consequence, the cornea may be squeezed in a manner that will cause wrinkles to be created through the cornea and predominantly over the posterior surface of the cornea. These wrinkles can then cause an undesirable dispersion and degradation of the laser beam as it passes through the cornea. Several other consequences may also result. For one, when the beam is degraded in this way, it can happen that the energy required for a procedure may need to be increased as much as five-fold to compensate for the degradation. Further, when the beam is dispersed, it becomes increasingly difficult to reach the Laser Induced Optical Breakdown (LIOB) threshold at the desired location, and non-targeted tissue can be unintentionally damaged.

In light of the above, it is an object of the present invention to dock the eye in preparation for a laser surgical procedure by using suction to draw the eye upwards, to thereby avoid creating wrinkles in the cornea of an eye. Another object of the present invention is to minimize the distortion and degradation of a laser beam as it travels through the cornea to perform an ophthalmic procedure on tissue in the eye, behind the cornea. Still another object of the present invention is to provide a docking device that includes a vacuum that can be adjusted to different levels to allow for a selective distortion of the cornea. Yet another object of the present invention is to provide a device and method for docking a patient's eye with an interface using suction that is easy to use, relatively simple to manufacture, and comparatively cost effective.

### SUMMARY OF THE INVENTION

In accordance with the present invention, a system and method are provided for docking an eye with an interface device that will avoid or minimize the creation of wrinkles on the posterior surface of the cornea of the human eye during a stabilization of the eye. For the present invention, this is accomplished by drawing the anterior surface of the eye into contact with a contact lens to effectively spread the cornea. This is done instead of pressing the contact lens downward onto the eye in a manner that will likely cause wrinkles in the cornea and on the posterior surface of the cornea. The object here is to avoid the wrinkles that could otherwise distort or degrade a laser beam.

Structurally, the system of the present invention includes an interface device that is constructed by securely affixing a base member and an attachment member to one another. For this combination, the base member is formed as a hollow, tapered cylinder having a proximal end and a distal end, with the proximal end having a larger diameter than the distal end. The base member defines a longitudinal axis, and a contact lens is located at the distal end on the longitudinal axis. For the present invention, the contact lens is transparent and it has a concave distal surface with a radius of curvature, R_{c}. As envisioned for the present invention, the contact lens may be either a rigid contact lens, a semi-rigid contact lens, or a non-rigid (i.e. soft) contact lens. The base member may also include an annular rib that is formed onto the outer surface of the base member near the proximal end. When so included, the annular rib projects outward from the outer surface, is centered on the axis, and extends completely around the base member.

Similar to the base member, the attachment member is a hollow, tapered cylinder with an open proximal end and an open distal end. Like the base-member, the attachment member defines a longitudinal axis and has a greater diameter at its proximal end than at its distal end. Importantly, the periphery of the open distal end establishes a continuous abutment that surrounds an opening, and is centered on the longitudinal axis. This opening is preferably circular, but any appropriate symmetrical or asymmetrical shape will suffice for use with the present invention. An annular groove is formed onto the interior surface of the attachment member that is centered on the longitudinal axis and is located proximal the abutment. A vacuum port is provided to establish fluid communication between the outer surface of the attachment member and the groove. When an annular rib is included on the base member, an annular detent is also included on the inner surface of the attachment member proximal to the groove. This detent is substantially parallel to the groove and is configured to receive the annular rib.

For a construction of the interface device, the base member and the attachment member are securely affixed to one another to establish a vacuum channel between the inner surface of the attachment member and the outer surface of the base member. When a rib and detent are included, the detent of the attachment member receives the rib of the base member to secure the base member to the attachment member with an airtight seal. Thus, the vacuum channel extends from the groove to the distal end of the attachment member around the base member. A vacuum pump can then be connected in fluid communication with the vacuum channel via the vacuum port and the groove.

In an operation of the present invention, the interface device is positioned onto the eye of a patient. Preferably, the initial contact between the eye and the interface device is made by the continuous abutment of the attachment member contacting the sclera of the eye. When in contact with the sclera, the continuous abutment effectively surrounds the cornea. As a consequence of this contact, an air pocket is established between the distal surface of the contact lens and the anterior surface of the eye that is bounded by the continuous abutment. Contact is not established, however between the contact lens and the anterior surface of the eye. Consequently, the radius of curvature of the anterior surface of the eye remains anatomically normal with a radius of curvature, Rₐ. Importantly, this Rₐ is smaller than the radius of curvature of the distal surface of the contact lens, (i.e. Rₐ<R_{c}).

For the present invention, the air pocket is established in fluid communication with the vacuum channel and the vacuum port. The vacuum pump, which is attached to the vacuum port can then be activated to suction air from the air pocket and bring the anterior surface of the eye into contact with the distal surface of the contact lens. It should be noted that the vacuum pump can be operated to establish various vacuum levels. By adjusting the vacuum level, the portion of the anterior surface of the eye that is drawn against the contact lens can be controlled and varied at any time.

Once contact between the eye and the contact lens has been established, the radius of curvature of the anterior surface of the eye, Rₐ, becomes equal to the radius of curvature of the distal surface of the contact lens (i.e. Rₐ=R_{c}) at their interface. Consequently, the cornea is spread outwardly as the posterior surface of the cornea is effectively stretched. At this point, the cornea, or a predetermined portion of the cornea, is appropriately docked, or stabilized, and the occurrence of wrinkles has been avoided or minimized. The ophthalmic procedure can then be conducted without having to account for beam distortions or degradations caused by wrinkles in the posterior region of the eye. Upon completion of the ophthalmic procedure, the vacuum pump is turned off, and the interface device can be lifted off of the eye.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a view of the interface device in an operational environment with the interface device positioned on the eye of a patient;

FIG. 2A is an exploded cross-sectional view of the base member and the attachment member of the interface device of the present invention as seen along the line 2-2 in FIG. 1;

FIG. 2B is a cross-sectional view of the interface device as seen in FIG. 2A with the base member joined with the attachment member;

FIG. 3A is a cross-sectional view of the interface device as shown in FIG. 2B, with the interface device positioned on the eye of a patient to establish an air pocket between the device and the eye prior to the creation of a vacuum in the air pocket; and

FIG. 3B is a cross-sectional view of the interface device and eye as seen in FIG. 3A after the creation of a vacuum in the air pocket.

### DESCRIPTION OF PREFERRED EMBODIMENTS

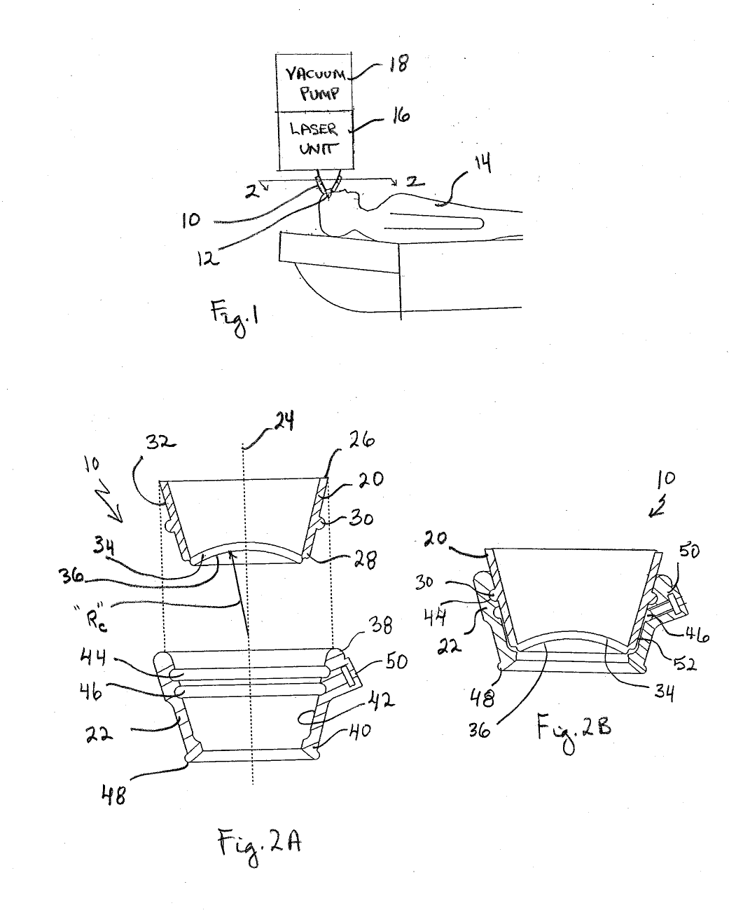

Referring initially to FIG. 1, an interface device in accordance with the present invention is shown in its operational environment and is designated 10. As shown, the interface device 10 is intended to be used for stabilizing an eye 12 of a patient 14 during ophthalmic surgery. More specifically, stabilization of the eye 12 is required for the use of a laser unit 16 during ophthalmic surgery. In accordance with the present invention, this stabilization is accomplished by employing a vacuum pump 18 that suctions (i.e. draws) the eye 12 into contact with the interface device 10.

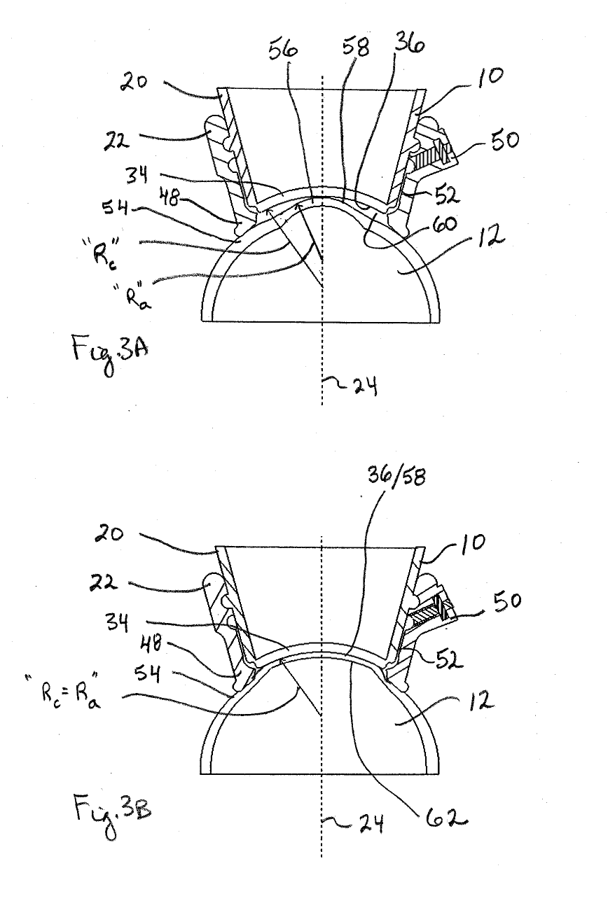

Referring now to FIG. 2A, an interface device 10 for the present invention is shown with its major constituent parts separated from each other. As shown, these parts include a base member 20 and an attachment member 22 which are both oriented on a common axis 24. In detail, the base member 20 is shown to be hollow and cylindrical in shape, and it is shown to have a proximal end 26 and a distal end 28. Further, the base member 20 is shown to be tapered with a decreasing cross section in the distal direction. An annular rib 30 is shown formed on the outer surface 32 of the base member 20, and the rib 30 is shown centered on the axis 24. Also, FIG. 2A shows that a contact lens 34 covers the distal end 28 of the base member 20, and that the contact lens 34 is formed with a concave distal surface 36 having radius of curvature “R_{c}”. Preferably, the contact lens 34 is made of a transparent material, such as a clear plastic. Depending on the particular material that is used for its construction, the contact lens 34 may be either rigid, semi-rigid or soft.

The attachment member 22 is shown in FIG. 2A to have a proximal end 38 and a distal end 40. Similar to the base member 20, the attachment member 22 is hollow and it also is tapered with a decreasing cross section in the distal direction. FIG. 2A further shows that the inner surface 42 of the attachment member 22 is formed with an annular shaped detent 44 and an annular shaped groove 46. The detent 44 and the groove 46 are shown in FIG. 2A to be substantially parallel to each other, with both being centered on the axis 24. Also shown in FIG. 2A are an abutment 48 that is established at the distal end 40 of the attachment member 22, and a vacuum port 50 that is formed on the attachment member 22 in fluid communication with the groove 46.

In FIG. 2B, the base member 20 and the attachment member 22 are shown joined together to establish the interface device 10 of the present invention. Specifically, the rib 30 of the base member 20 is shown seated in the detent 44 of the attachment member 22 to create an airtight seal. As will be appreciated by the skilled artisan, the base member 20 and the attachment member 22 may be joined together to create such an airtight seal in any of several different ways, all well known in the pertinent art. In any event, it will be important that the device 10 have a vacuum channel 52 that is in fluid communication with the vacuum port 50. As shown in FIG. 2B, the vacuum channel 52 is established between the outer surface 32 of the base member 20 and the inner surface 42 of the attachment member 22. Further, the vacuum channel 52 extends in a distal direction around the base member 20, from the airtight seal that is created when the rib 30 is seated in the detent 44 to the concave distal surface 36 of the contact lens 34.

In an operation of the interface device 10 of the present invention, the device 10 is positioned against the eye 12 of a patient 14, substantially as shown in FIG. 3A. In detail, the abutment 48 of attachment member 22 is preferably positioned against the sclera 54 of the eye 12 to surround the cornea 56. Depending on the particular procedure to be performed, however, the abutment 48 could as well be positioned directly against selected portions of the anterior surface 58 of the cornea 56. Importantly, regardless of how the device 10 may be positioned with the abutment 48 in contact with the eye 12, the concave distal surface 36 of the contact lens 34 will not contact the eye 12. Instead, the concave distal surface 36 of the contact lens 34 will be initially distanced from the anterior surface 58 of the cornea 56. Thus, an air pocket 60 is established between the contact lens 34 and the eye 12. It is also an important consideration at this point, to note that the normal, anatomical radius of curvature “Rₐ” of the anterior surface 58 of the cornea 56 will be less than the radius of curvature “R_{c}” of the contact lens 34. This difference, however, is preferably minimal as it is desirable to put as little stress as possible on the cornea 56. Consequently, in the event, the periphery of the cornea 56 will be farther from the concave distal surface 36 of the contact lens 34 than will be the center of the cornea 56.

After the interface device 10 has been positioned on the eye 12 as disclosed above, the vacuum pump 18 can be activated to create a partial vacuum in the air pocket 60. More specifically, via a fluid connection between the vacuum pump 18 and the vacuum port 50, the vacuum pump 18 can be selectively operated, at different vacuum levels, to draw different proportional areas of the anterior surface 58 of the cornea 56 into contact with the concave distal surface 36 of the contact lens 34. Stated differently, as the vacuum level is increased inside the air pocket 60, the area of contact between the anterior surface 58 of the cornea 56 and the concave distal surface 36 of the contact lens 34 will become proportionately larger. As shown in FIG. 3B, substantially all of the anterior surface 58 of cornea 56 is in contact with the concave distal surface 36 of the contact lens 34 (i.e. Rₐ becomes essentially equal to R_{c}).

As intended for the present invention, when a partial vacuum has been created in the air pocket 60 by the vacuum pump 18, the cornea 56 is drawn into contact with the contact lens 34. A consequence here is that the cornea 56, and its posterior surface 62, are generally spread out into a partially flattened configuration. A further consequence of this is that wrinkles (not shown), that might otherwise be created on the posterior surface 62 of the cornea 56 during a stabilization of the eye 12, are avoided.

While the particular System and Method for Docking a Cornea with a Patient Interface Using Suction as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. An interface device for stabilizing an eye during ophthalmic surgery which comprises:
a cylindrical-shaped base member defining a longitudinal axis and having a proximal end and a distal end;
a contact lens centered on the axis and attached to the distal end of the base member, the contact lens having a concave distal surface with a radius of curvature “R_{c}”;
a hollow, cylindrical-shaped attachment member securely affixed to the base member to establish a vacuum channel therebetween;
a continuous abutment formed on the attachment member to surround the contact lens and create an air pocket between the concave distal surface of the contact lens and the anterior surface of the eye when the abutment is positioned against the sclera of the eye, wherein the anterior surface of the eye has a radius of curvature Rₐ, and wherein R_{c} is greater than Rₐ (R_{c}>Rₐ); and
a vacuum pump connected in fluid communication with the vacuum channel to create a vacuum in the air pocket for drawing the anterior surface of the eye into contact with the concave distal surface of the contact lens to stabilize the eye.

2. A device as recited in claim 1 wherein the base member is formed with an annular rib centered on the longitudinal axis of the base member, proximal the contact lens, and wherein the attachment member is formed with an annular detent centered on the longitudinal axis of the attachment member, and wherein the detent is dimensioned to receive the rib of the base member for affixing the attachment member to the base member.

3. A device as recited in claim 1 wherein the vacuum pump is operable at a plurality of different vacuum levels to selectively establish a vacuum level in the air pocket.

4. A device as recited in claim 3 wherein the vacuum level is set to establish a predetermined contact area between the concave distal surface of contact lens and the anterior surface of the eye.

5. A device as recited in claim 4 wherein Rₐ is selectively increased in response to an increase in the vacuum level in the air pocket.

6. A device as recited in claim 5 wherein R_{c} is equal to Rₐ (R_{c}=Rₐ) when the anterior surface of the eye is in contact with the concave surface of the contact lens.

7. A device as recited in claim 1 further comprising a vacuum port formed on the attachment member for connection with the vacuum pump.

8. A device as recited in claim 1 wherein the contact lens is selected from a group comprising a rigid contact lens, a semi-rigid contact lens, and a non-rigid contact lens.

9. A method for manufacturing and using an interface device during ophthalmic surgery which comprises the steps of:
providing a cylindrical-shaped base member defining a longitudinal axis and having a proximal end and a distal end;
attaching a contact lens to the distal end of the base member, wherein the contact lens is centered on the axis and has a concave distal surface with a radius of curvature “R_{c}”;
affixing a hollow, cylindrical-shaped attachment member to the base member to establish a vacuum channel therebetween;
forming a continuous abutment on the attachment member to surround the contact lens and create an air pocket between the concave distal surface of the contact lens and the anterior surface of the eye when the abutment is positioned against the sclera of the eye, wherein the anterior surface of the eye has a radius of curvature Rₐ, and wherein R_{c} is greater than Rₐ (R_{c}>Rₐ); and
connecting a vacuum pump in fluid communication with the vacuum channel to create a vacuum in the air pocket for drawing the anterior surface of the eye into contact with the concave distal surface of the contact lens to stabilize the eye.

10. A method as recited in claim 9 further comprising the step of operating the vacuum pump to selectively establish a vacuum level in the air pocket.

11. A method as recited in claim 10 further comprising the step of setting the vacuum level to establish a predetermined contact area between the concave distal surface of contact lens and the anterior surface of the eye.

12. A method as recited in claim 11 wherein Rₐ is selectively increased in response to an increase in the vacuum level in the air pocket.

13. A method as recited in claim 12 wherein R_{c} is equal to Rₐ (R_{c}=Rₐ) where the anterior surface of the eye is in contact with the concave surface of the contact lens.

14. A method as recited in claim 9 further comprising the step of forming a vacuum port on the attachment member for connection with the vacuum pump.

15. A method as recited in claim 9 wherein the contact lens is made of a transparent material and is selected from a group comprising a rigid contact lens, a semi-rigid contact lens, and a non-rigid contact lens.

16. A method as recited in claim 9 further comprising the steps of:
forming the base member with an annular rib centered on the longitudinal axis of the base member, proximal the contact lens; and
forming the attachment member with an annular detent centered on the longitudinal axis of the attachment member, wherein the detent is dimensioned to receive the rib of the base member for affixing the attachment member to the base member.

17. An interface device for stabilizing an eye during ophthalmic surgery which comprises:
a cylindrical-shaped base member having an outer surface and a closed distal end, wherein the base member defines a longitudinal axis and the distal end of the base member is formed as a contact lens having a concave distal surface centered on the axis;
a hollow, cylindrical shaped attachment member having an open distal end and an inner surface, wherein the attachment member defines a longitudinal axis and is formed with a continuous abutment centered on the axis at the distal end of the attachment member;
a means for affixing the base member to the attachment member to establish a vacuum channel between the outer surface of the base member and the inner surface of the attachment member, with the abutment of the attachment member extended distally beyond the concave distal surface of the contact lens;
a vacuum port formed on the attachment member for fluid communication with the vacuum channel;
a means for positioning the abutment of the attachment member against the sclera of the eye to create an air pocket between the concave distal surface of the contact lens and the anterior surface of the eye; and
a vacuum pump operable at a selected vacuum level, wherein the vacuum pump is connected with the vacuum port to create a vacuum in the air pocket, to draw the anterior surface of the eye into contact with the concave distal surface of the contact lens to stabilize the eye, wherein the vacuum level is selectively adjustable during ophthalmic surgery to establish a predetermined contact area between the concave distal surface of the contact lens and the eye.

18. A device as recited in claim 17 wherein the concave distal surface of the contact lens has a radius of curvature R_{c} and the anterior surface of the eye has a radius of curvature Rₐ, and wherein R_{c} is greater than Rₐ (R_{c}>Rₐ).

19. A device as recited in claim 17 wherein Rₐ is selectively increased in response to an increase in the vacuum level in the air pocket.

20. A device as recited in claim 17 further comprising:
an annular rib formed on the outer surface of the base member and centered on the longitudinal axis of the base member, proximal the contact lens;
an annular groove formed on the inner surface of the attachment member and centered on the longitudinal axis of the attachment member at a location proximal the abutment; and
an annular detent formed on the inner surface of the attachment member proximal the groove and parallel thereto, for engagement of the detent with the rib of the base member to establish a vacuum channel extending distally from the groove between the outer surface of the base member and the inner surface of the attachment member, with the abutment of the attachment member extended distally beyond the concave distal surface of the base member.

21. An interface device for stabilizing an eye during ophthalmic surgery which comprises:
a base member defining a longitudinal axis and having a proximal end and a distal end;
a contact lens centered on the axis and attached to the distal end of the base member, wherein the contact lens has a periphery surrounding a concave distal surface, and the concave distal surface has a radius of curvature “R_{c}”;
a continuous abutment formed on the base member to establish a vacuum channel between the abutment and the periphery of the contact lens, wherein the abutment creates an air pocket between the concave distal surface of the contact lens and the anterior surface of the eye when the abutment is positioned against the eye, and wherein the anterior surface of the eye has a radius of curvature Rₐ, with R_{c} being greater than Rₐ (R_{c}>Rₐ); and
a vacuum pump connected in fluid communication with the vacuum channel to create a vacuum in the air pocket for drawing the anterior surface of the eye into contact with the concave distal surface of the contact lens to stabilize the eye.

22. A device as recited in claim 21 wherein the vacuum pump is operable at a plurality of different vacuum levels to selectively establish a vacuum level in the air pocket, and the vacuum level is set to establish a predetermined contact area between the concave distal surface of contact lens and the anterior surface of the eye.

23. A device as recited in claim 22 wherein Rₐ is selectively increased in response to an increase in the vacuum level in the air pocket, and wherein R_{c} is equal to Rₐ (R_{c}=Rₐ) when the anterior surface of the eye is in contact with the concave surface of the contact lens.
